(19) United States
(12) Patent Application Publication
(10) Pub. No.: US 2010/0152544 A1
(43) Pub. Date: Jun. 17, 2010

(54) CARDIAC MONITORING SYSTEM AND METHOD

(75) Inventors: James Whitt Weaver, Columbia, SC (US); Stephen Brian Turner, Cheltenham (GB)

Correspondence Address: ADAMS INTELLECTUAL PROPERTY LAW, Suite 2350 Charlotte Plaza, 201 South College Street, CHARLOTTE, NC, 28244, US

(73) Assignee: SPECTRUM MEDICAL LIMITED, Gloucester (GB)

(21) Appl. No.: 12/621,016

(22) Filed: Nov. 18, 2009

Related U.S. Application Data
(60) Provisional application No. 61/116,148, filed on Nov. 19, 2008.
(60) Provisional application No. 61/119,049, filed on Dec. 2, 2008.

(51) Int. Cl. A61B 5/00; G06Q 10/00; G06Q 50/00; G06F 3/048; G06F 15/16

(52) U.S. Cl. 600/300; 705/2; 715/764; 709/217

(57) ABSTRACT

A cardiac monitoring system and method for configuring a case template including selecting critical parameters to be monitored during a procedure, entering patient-specific details for computing patient values for determining control limits for the critical parameters, setting control limits for each of the selected critical parameters, monitoring parametric data in real-time against the set control limits, capturing and recording event parametric data related to control limit breach events, and charting the event parametric data corresponding with the timing of the control limit breach event.

### CROSS-REFERENCE TO RELATED APPLICATIONS

This application claims priority to U.S. Provisional Patent Application No. 61/116,148 filed Nov. 19, 2008 and entitled “TEMPLATE FOR CARDIAC MONITORING PROCESS”, and U.S. Provisional Patent Application No. 61/119,049 filed Dec. 2, 2008 and entitled “DUAL FLOW CARDIAC MONITORING SYSTEM”, the contents of which are incorporated by reference herein.

### FIELD OF THE INVENTION

The present invention provides a cardiac monitoring system and method for creating case templates having specific chart configurations that allow for the selection of critical physiological parameters and control limits, as well as monitoring data against pre-programmed control limits in real-time and recording and charting information corresponding with the timing of events to improve patient outcomes and quality of care.

### BACKGROUND OF THE INVENTION

Conventional cardiac monitoring apparatus require that the attending physician manually input desired parameters into the monitoring system. These desired parameters include a myriad of different variables and are fact specific in regards to the surgeon's preferences, the patient, and the type of surgery being performed. Selecting this data for each medical procedure is cumbersome, and in some instances, this data may even be entered incorrectly, thus exposing the patient to potential medical harm and the physician to potential liability. Accordingly, there remains a need for a procedure that can be repeatedly and accurately used for entering desired parameters for a medical procedure.

### BRIEF SUMMARY OF THE INVENTION

According to an embodiment of the invention, a method for configuring a case template in a cardiac monitoring system is provided including the steps of selecting critical parameters to be monitored during a procedure, entering patient-specific details for computing patient values for determining control limits for the critical parameters, and setting control limits for each of the selected critical parameters. In a further embodiment, the method includes the steps of entering names of personnel attending the procedure, entering per-operative lab values for the patient, entering priming-related data for a priming circuit for the procedure and calculating optimal fluid compositions based on the patient-specific details and pre-operative lab values, entering disposable items to be used during the procedure, entering equipment to be used during the procedure, and configuring event timers for the procedure corresponding to parametric data falling outside of the set control limits. In a still further embodiment, the method includes the steps of monitoring parametric data in real-time against the set control limits, providing an alarm for a control limit breach event, capturing and recording event parametric data related to the control limit breach event, charting the event parametric data corresponding with the timing of the control limit breach event, producing a perfusion chart record including the event parametric data, and saving the template according to an appropriate clinical or control profile.

Patient values include at least one of body mass index, body surface area, body surface area cardiac index, weight cardiac index, body surface area flow rate, and weight flow rate. The step of entering priming-related data includes selecting bypass factors for loading preset priming volumes and computing drug volumes from dosages. Monitored parametric data may include both static and continuous data, wherein static data is data provided to the monitoring system on an intermittent basis and continuous data is data collected at a user specified frequency.

According to other embodiments, the method further includes the steps of providing a graphical user interface including a display for displaying a plurality of soft keys for accessing a plurality of data pages each for at least one of entering, receiving, storing, arranging, manipulating, charting, generating, retrieving, viewing and reporting data, communicating with a hospital management system and downloading the perfusion chart record to a database for study to improve quality of care, categorizing the control limit breach event into one of a bypass, fluid, manual set point, cardioplegia, blood sample, personnel change, disposable change, equipment change, and flagged event, and providing a checklist for confirming that a set of predefined tasks have been completed prior to initiating monitoring the parametric data, wherein the predefined tasks include clock synchronization, entry of the patient-specific details and equipment checks. Critical parameters may be selected according to surgeon preferences, patient details and procedure type.

According to another embodiment of the invention, a cardiac monitoring system is provided including a graphical user interface for displaying a plurality of soft keys for accessing a plurality of data pages each for at least one of entering, receiving, storing, arranging, manipulating, charting, generating, retrieving, viewing and reporting data. The system further includes a storage device including a computer-readable medium containing instructions for configuring a cardiac monitoring procedure template that includes configuration functions for selecting critical parameters to be monitored during the procedure, entering patient-specific details for computing patient values for determining control limits for the critical parameters, setting control limits for each of the selected critical parameters, monitoring parametric data in real-time against the set control limits, providing an alarm for a control limit breach event, capturing and recording event parametric data related to the control limit breach event, charting the event parametric data corresponding with the timing of the control limit breach event, and producing a perfusion chart record including the event parametric data.

According to another embodiment, the configuration functions further include at least one of saving the template according to an appropriate clinical or control profile, entering names of personnel attending the procedure, entering per-operative lab values for the patient, entering priming-related data for a priming circuit for the procedure and calculating optimal fluid compositions based on the patient-specific details and pre-operative lab values, entering disposable items to be used during the procedure, entering equipment to be used during the procedure, and configuring event timers for the procedure corresponding to parametric data falling outside of the set control limits.

Entering priming-related data includes selecting bypass factors for loading preset priming volumes and computing drug volumes from dosages. Patient values include at least one of body mass index, body surface area, body surface area cardiac index, weight cardiac index, body surface area flow rate, and weight flow rate. Monitored parametric data includes both static and continuous data, wherein static data is data provided to the monitoring system on an intermittent basis and continuous data is data collected at a user specified frequency.

According to another embodiment, the system further includes a hospital management server in communication with the cardiac monitoring system for downloading the perfusion chart record thereto to improve quality of care.

According to another embodiment, the configuration functions further include categorizing the control limit breach event into one of a bypass, fluid, manual set point, cardioplegia, blood sample, personnel change, disposable change, equipment change, and flagged event, and providing a checklist for confirming that a set of predefined tasks have been completed prior to initiating monitoring the parametric data, wherein the predefined tasks include clock synchronization, entry of the patient-specific details and equipment checks. Critical parameters may be selected according to surgeon preferences, patient details and procedure type.

### BRIEF DESCRIPTION OF THE FIGURES

Features, aspects and advantages of the present invention are better understood when the following detailed description of the invention is read with reference to the accompanying drawings, in which:

FIG. 1 is a schematic diagram illustrating a graphical using interface including a plurality of soft keys having predetermined assigned functions; and

FIG. 2 is a flow diagram illustrating a cardiac monitoring method according to an embodiment of the invention.

### DETAILED DESCRIPTION OF THE INVENTION

The present invention will now be described more fully hereinafter with reference to the accompanying figures in which exemplary embodiments of the invention are shown. However, the invention may be embodied in many different forms and should not be construed as limited to the representative embodiments set forth herein. The exemplary embodiments are provided so that this disclosure will be both thorough and complete, and will fully convey the scope of the invention and enable one of ordinary skill in the art to make, use and practice the invention.

### System Overview

Referring to the figures, the present invention is directed to a cardiac monitoring system and method for producing customizable templates with predetermined control limits, and capturing factual real-time data to improve patient outcomes and to protect clinicians from discrepancies in charting when events occur during a procedure. In particular applications, the monitoring system and method may be utilized during cardiac surgery and respiratory Extracorporeal Membrane Oxygenation (ECMO) procedures, among other procedures. The electronic charting system and method provided herein are event driven and customizable to meet the needs of the various configurations and specifications of each patient or clinician.

The cardiac monitoring system and method further provides for the creation of templates for surgeon, patient or procedure protocol that includes specific chart configurations that allow for the selection of critical physiological parameters and control limits. Data is monitored against these pre-programmed control limits in real-time, and in the event of a control limit breach, the monitoring system records and charts information corresponding with the timing of the event and allows the user to enter additional information. Collected data may be downloaded to a database to be studied to improve quality of care. In an additional embodiment, the cardiac monitoring system provided herein is in communication with a hospital management system.

The cardiac monitoring system and method provided herein combines electronic data collection with the real-time non-invasive measurement of arterial and venous saturation, venous and arterial flow, flow differential, and venous and arterial emboli detection.

As will be understood and appreciated by those skilled in the art, the cardiac monitoring system and method of the present invention may be embodied as a tool, a method, a data processing system, a computer program product, and a web-based software application. Accordingly, the invention may take the form of an embodiment combining software and hardware aspects. Furthermore, the invention may take the form of a computer program product on a computer-readable storage medium having computer-readable program instructions (e.g., software application) embodied in the storage medium. More particularly, the invention may take the form of web-implemented computer software. Any suitable computer-readable storage medium known to those skilled in the art may be utilized including, but not limited to, hard disks, CD/DVD-ROMs, optical storage devices, magnetic storage devices and flash memory.

The invention is described below with reference to figures illustrating methods, apparatuses (i.e., systems) and computer program products according to preferred embodiments of the invention. It will be understood that each block of the figures, and combinations of blocks in the figures, respectively, can be implemented by computer program instructions. These computer program instructions may be loaded onto a computer, server, special purpose computer or other programmable data processing apparatus to produce a machine, such that the instructions that execute on the computer or other programmable data processing apparatus create a means for implementing the functions specified in the figures.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flow and block diagrams. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the figures.

Accordingly, blocks of the figures support combinations for performing the specified functions, combinations of steps for performing the specified functions and program instructions for performing the specified functions. It will also be understood that each block of the figures, and combinations of blocks in the figures, can be implemented by special purpose hardware-based computer systems that perform the specified functions or steps, or combinations of special purpose hardware and computer instructions.

### System Architecture

The system for implementing the modules and method described in detail below includes a computer or other interface device (i.e. Graphical User Interface (GUI) pre-loaded with the software application and/or optionally in communication with a data collection and management system operating on a server. Access to the server may be web-based. The system is configured for at least entering, receiving, storing, arranging, manipulating, charting, generating, accessing, retrieving and reporting data. In a preferred embodiment, the computer is a portable user interface such as a monitor having a display with touch-screen and display overlay functionality, and wireless communication connectivity.

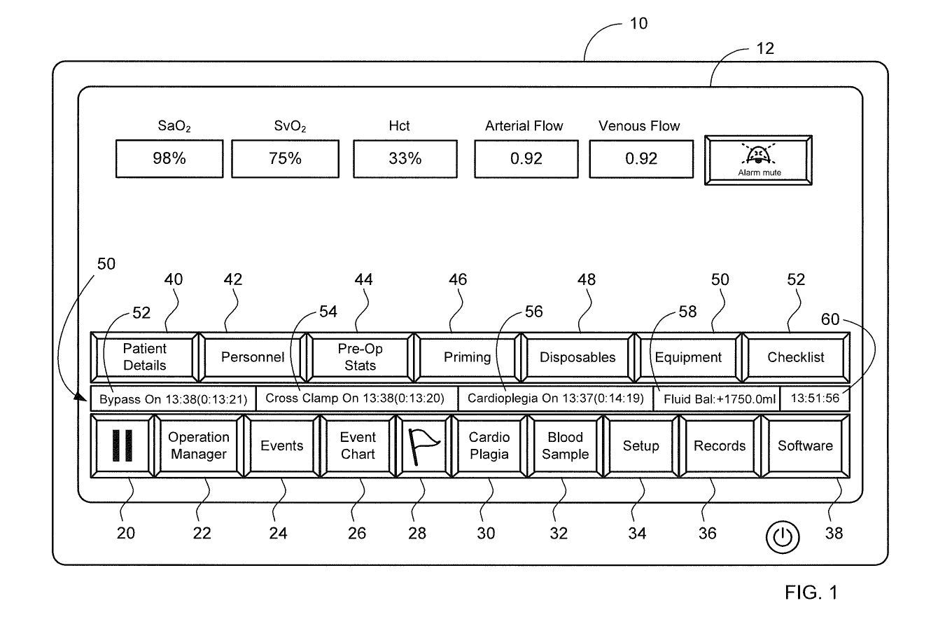

Referring to FIG. 1, a schematic diagram illustrating the cardiac monitoring system Graphical User Interface (GUI) is shown as monitor 10 having display screen 12 with touch screen functionality and the ability to present graphical icons, visual indicators and special graphical elements. The GUI is preferably an electronic charting interface for perfusion and ECMO applications. The monitoring system is preferably configured to communicate with external devices.

The system includes an operating system and a processor that communicates with other elements within the system via a system interface or bus. Also included in the system is the display device/input device (i.e. monitor) for receiving, inputting and displaying data. The system further includes memory, which preferably includes both read only memory (ROM) and random access memory (RAM). The system's ROM is used to store a basic input/output system (BIOS), containing the basic routines that help to transfer information between elements within the system.

In addition, the system includes at least one storage device, such as a hard disk drive, CD/DVD Rom drive or optical disk drive for storing information on various computer-readable media, such as a hard disk, removable magnetic disk, CD/DVD-ROM disk or flash memory. As will be appreciated by those skilled in the art, each of these storage devices is connected to the system bus by an appropriate interface. The storage devices and their associated computer-readable media provide nonvolatile storage. System modules are stored by the various storage devices and within RAM. The system modules are used to control certain aspects of the operation of the system with the assistance of the processor and an operating system.

### System Modules and Methods

The cardiac monitoring system includes various modules for providing functionality for at least creating customizable templates including parameter control limits, entering data, recording and managing data, recording events and event-related data, and creating charts and perfusion records for events, among other functions. Created templates are saved and selected based upon the appropriate clinical or control profile, and charts are configured to record a pre-selected range of clinical parameters. Control limits are selected for each of the parameters selected during a chart configuration process, and individual limits may be further defined for both arterial and venous blood samples in a particular embodiment.

Using established control limits, the monitoring system reports event alarms for data falling outside (i.e. above or below) of the control limits considered to be critical information labeled as an ‘Event’. Event alarms trigger the creation of a perfusion chart record that includes the plotting of parametric data. Both static and continuous data is recorded and managed. “Static data” is considered to be data provided to the monitoring system on an intermittent basis. “Continuous data” is considered to be data collected at a user specified frequency. Exception ‘Events’ preferably trigger the collection of all continuous data regardless of frequency.

The cardiac monitoring system further has the additional functionality to extend the utility of a perfusion chart by generating post-operative analysis, which relies on analyzing data that either falls within or out of the user defined control limits. The user has the ability to select a parameter for analyses and select from a predefined list of computations. In a particular embodiment, only parameters that have been pre-selected and fall outside of the control limits are reported in a perfusion record, as data within the control limits is not considered an ‘Event’.

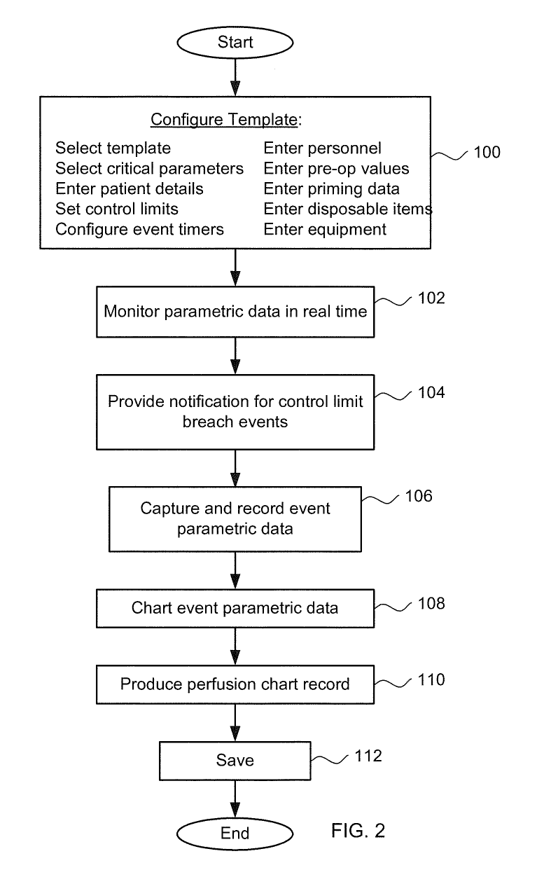

Referring to FIG. 2, a flow diagram illustrating the cardiac monitoring method according to the preferred embodiment of the invention is shown. The method includes the steps for configuring a case template (Step 100) including: selecting a template; selecting critical parameters to be monitored during a procedure; entering patient details; setting control limits for the parameters; entering personnel; entering pre-operative lab values; entering priming-related data; entering disposable items; entering equipment; and configuring event timers. Once a template is configured, the method further includes monitoring parametric data in real-time against the set control limits (Step 102), providing notification for control limit breach events (Step 104), capturing and recording event parametric data related to the control limit breach events (Step 106), charting the event parametric data corresponding with the timing of the control limit breach events (Step 108), producing a perfusion chart record including the event parametric data (Step 110), and saving the template according to an appropriate clinical or control profile (Step 112).

Referring again to FIG. 1, the various functions of the cardiac monitoring system are accessed/activated through simulated buttons or “soft keys”, referred to herein collectively as “keys”, having predetermined assigned functions. Keys are preferably tapped with a finger or stylus for activation and are shown remaining in a presses state when activated. Various status boxes are also displayed on display 12 including timers, flow rates, etc. System control keys are shown in FIG. 1 positioned along the lower region of display 12 and include ‘Status’ key 20, ‘Operation Manager’ key 22, ‘Events’ key 24, ‘Event Chart’ key 26, ‘Add Event’ key 28, ‘Cardioplegia’ key 30, ‘Blood Sample’ key 32, ‘Setup’ key 34, ‘Records’ key 36, and ‘Software’ key 38. Sub-keys for accessing various data pages are accessed through the control keys. The functions of the various keys and pages displayed are described below in detail.

System control ‘Status’ key 20 displays an icon and/or text indicating the next state the application can and will enter if the key is pressed. ‘Status’ key 20 is preferably always enabled. The current status is the state prior to the current displayed icon and text. The initial state upon starting is indicated as ‘Start’ on the display. States may include ‘Ready’, ‘Started’, ‘Paused’ and ‘Review’, among others. Exemplary state logic includes a ‘Ready’ state displaying a play icon with the next state being ‘Started’, ‘Started’ displaying a pause icon with the next state being ‘Paused’ or ‘Review’, ‘Paused’displaying a flashing pause icon with the next state being ‘Started’ or ‘Review’, and ‘Review’ flashing a save icon with the next state being ‘Ready’. Pressing ‘Status’ key 20 in either the ‘Ready’ or ‘Review’ state will change the state without prompt. Pressing ‘Status’ key 20 in either the ‘Started’ or ‘Paused’ state will display a dialog with options as to which state to enter. ‘Cancel’ is available to return to the current state. Changes from the ‘Ready’ state to the ‘Started’ state cause the active main region page to switch to ‘Events’.

‘Operation Manager’ key 22 opens an ‘Operation Manager’ page in the main region of display 12. When the ‘Operation Manager’ page is displayed, key 22 is shown in a pressed state. ‘Operation Manager’ key 22 is preferably always enabled. Referring to FIG. 2, pressing key 22 displays a plurality of sub-keys corresponding to sub-pages displayed on the main display. As shown, the sub-keys/sub-pages include ‘Patient Details’ key 40, ‘Personnel’ key 42, ‘Pre-Op Stats’ key 44, ‘Priming’ key 46, ‘Disposables’ key 48, ‘Equipment’ key 50, and ‘Checklists’ key 52. Sub-pages include various fields for being populated with data arranged in sections, rows and columns. Data entered into these fields is saved and may be utilized by other system modules.

‘Patient Details’ key 40 opens a ‘Patient Details’ sub-page for entering patient specific details other than pre-operative and post-operative statistics. The page is preferably the first page displayed when the cardiac monitoring system is launched. The page includes fields for entering patient data including, but not limited to, a case number or identifier, patient name, birth date, weight, height, gender and age. Data may further include selecting a blood type from a provided list, calculating and displaying the patient's Body Mass Index (BMI) from the entered height and weight, calculating and displaying the patient's body Body Surface Area (BSA) from a list of pre-loaded calculations (e.g. Mosteller, Haycock, Du Bois & Du Bois, Gehan E A, Boyd's Formula, and National Cancer Institute computational formulas based on weight and height), the patient's BSA Cardiac index (L/m²/min) and weight Cardiac Index (ml/kg/min), and BSA and weight flow rates. Computed values are updated as fields are updated. For example, an update of patient weight, height or corresponding cardiac index updates the flow rate as the product of the cardiac index and BSA. Individual patient tables are further provided for entering and recording data such as ‘Diagnosis’, ‘Procedure’, and ‘Allergy/Caution’ tables.

‘Personnel’ key 42 opens a ‘Personnel’ sub-page for entering the names of attending personnel during a case. For example, names may be entered for each attending surgeon, anesthesiologist, nurse, perfusionist, perfusion assistant, etc. Date and time stamps are recorded for each change made to the personnel page. Events are added denoting the time the change was made. Before a case may begin and personnel data entered, a template must first be chosen that loads configuration settings and continuous data along with control limits for each value as described in detail below. If a user presses the ‘Start’ button to begin a case without previously selecting an input configuration template, a prompt is provided for first selecting a template.

Pre-Op Stats' key 44 opens a ‘Pre-Op Statistics’ sup-page for entering items such as lab values. Desired values for entry may include, but are not limited to, Hematocrit/Hemoglobin, Fibrinogen (mg/dl), COP (mmHg), and Platelets/μl.

‘Priming’ key 46 opens a ‘Priming’ sub-page for entering data related to the priming circuit, such as volume and composition. The ‘Priming’ sub-page may also function as a tool for calculating optimal fluid compositions for given patient statistics such as weight as well as the fixed pre-operative statistics. The ‘Priming’ sub-page may include sections for ‘Bypass Factors’, ‘Fluids’ and ‘Computed Values’. ‘Bypass Factors’ are used to load preset priming volumes, predict lab values after hemodilution, compute priming fluids to achieve target lab values, and compute drug volumes from dosages, among other uses. Changes made to any of the options in this group cause calculated values and priming volumes to be re-computed once a target value has been entered.

‘Bypass Factors’ of the ‘Priming’ sub-page include circuit volume presets, blood volume factors, input Hct/Hb values, target Hct/Hb values, input Fibrinogen values, target Fibrinogen values, input albumin values, target COP values, and drugs. ‘Drugs’ preferably includes fluids added in the fluids table set as drugs. Drug volumes are calculated in the fluid table based on the patient weight and dosage. Changes to weight automatically update the drug volumes computed in the fluid table and the fluid balance and predictions. The system includes a standard calculator that is preferably always enabled for use as needed. The ‘Fluid’ table includes fixed fluids such as crystalloid, albumin, packed RBCs, FFP and anesthesia. Names and values are saved. Conversion factors for converting units to those desired to be displayed are provided. ‘Computed Values’ are preferably read-only with no input mode. ‘Computed Values’ include predicted Hct/Hb, predicted Fibrinogen, predicted platelet count, and total priming volume, with the algorithms for predicting each provided below.

The algorithm for predicting Hct/Hb is provided as follows and is preferably displayed with 1-digit past the decimal place followed by ‘%’:

Total blood volume (TBV) is computed as follows:

TBV=Blood Volume Factor (ml/kg)×Weight (kg)× 1/1000 (L/ml)

The total volume (TV) after prime is computed as follows:

TV=TBV+PV (PV is priming volume in L)

The total red blood cells (tRBC) in the patient are computed as follows:

tRBC=TBV(L)×pHct(%)/100 (pHct is the pre-operative Hematocrit value)

The total RBC added to prime (pRBC) is computed as follows:

pRBC=inRBC(L)×inHct(%)/100 (in RBC is packed RBCs or whatever form RBCs are give to patient and in Hct is the Hematocrit value of the added fluid)

The predicted Hematocrit after priming (HCTnew) is computed as follows:

HCTnew=100×(tRBC+pRBC)/TV

The algorithm for predicting Fibrinogen is as follows and is preferably displayed with zero digits past the decimal followed by ‘gm/dl ’ and proceeded by “Predicted Fibrinogen=”:

Total blood volume (TBV) is computed as follows:

TBV=Blood Volume Factor (ml/kg)×Weight(kg)× 1/1000 (L/ml)

The total volume (TV) after prime is computed as follows:

TV=TBV+PV (PV is priming volume in L)

The total plasma (tPV) in the patient is computed as follows:

tPV=TBV(L)×(100−pHct(%))/100 (pHct is the pre-operative Hematocrit value)

The total Fibrinogen in the patient (tFib) in mg is computed as follows:

tFib=(tPV×10)×pFib(mg/dl) (pFib is the pre-operative Fibrinogen value)

The total volume on bypass other than RBC (nonRBCVol) is computed as follows:

nonRBCVol=TV×(100−HCTnew(%))/100 (HCTnew is predicted Hematocrit)

The Fibrinogen in the priming (pFib) in mg is computed as follows:

pFib=FFP(L)×inFib(mg/dl)×10 (dl/L) (FFP is volume of plasma and in Fib is the fibrinogen of the added plasma)

The predicted fibrinogen (Fibnew) in mg/dl is computed a follows:

Fibnew=(tFib+pFib)/(nonRBCVol×10 (dl/L))

The algorithm for predicting platelet count is provided as follows:

Total blood volume (TBV) is computed as follows:

TBV=Blood Volume Factor (ml/kg)×Weight(kg)× 1/1000 (L/ml)

The total volume (TV) after prime is computed as follows:

TV=TBV+PV (PV is priming volume in L)

The predicted platelet count (PlateletNew):

PlateletNew=pPlatelets×TBV/TV (pPlatelets is the pre-operative Platelet value #/mm³)

The total priming volume is the total of all fluids in the prime and is preferably displayed in units of ml.

‘Disposables’ key 38 opens a ‘Disposables’ sub-page for entering disposable items used during a case. The sub-page preferably shows the disposables currently in use and changes made to a case generate an event and the item and times when used are printed in the chart record. A plurality of ‘Disposable Presets’ are provided corresponding to patient type, examples of which include ‘Neonatal’, ‘Infant’, ‘Child’, ‘Small Adult’, ‘Adult’, ‘Large Adult’. Additional presets may be entered and saved. A ‘Disposable List’ is provided for displaying disposables and includes fields to be populated with the ‘Model/Size’ such as ‘Oxygenator’, ‘Venous Reservoir’, ‘Cardiotomy Reservoir’, ‘Tubing Pack’, ‘Arterial Filter’, ‘Centrifugal Pump’, ‘Auto-transfusion Set’, and ‘Cardioplegia Set’. Entry of a disposable ‘Model/Size’ prompts the opening of a second field for entering comments.

‘Equipment’ key 40 opens an ‘Equipment’ sub-page for entering descriptions of equipment used during the case. Changes made during a case generate an event and the item and times when changed are printed in the chart record. Equipment presets are provided, and presets may be added and saved. An equipment list is provided for displaying types of equipment used and includes fields for population, for example ‘Pump Console’, ‘Arterial Pump’, ‘Heater/Cooler’; Autotrans Device', ‘Cell Saver System’, ‘ACT Analyzer’, and ‘Blood Gas Analyzer’. Entry of equipment ‘Model/Size’ prompts the opening of a second field for entering comments.

‘Checklist’ key 42 opens a ‘Checklist’ sub-page for confirming that a set of predefined tasks has been completed before a case may be started. The monitoring system provides a notification of ‘Incomplete’ in a status bar if an attempt to start a case is made prior to completing the checklist. One fixed item in the checklist functions to synchronize the clocks. When this key is pressed, a time editing keypad appears followed by a date editing keypad. Other items on the list take the current time/date and fill the key with the text. Pressing a key with a time/date stamp presents a ‘YES/NO’ dialog to confirm removing the time/date stamp.

Referring again to the system control keys, ‘Events’ key 24 opens an ‘Events’ page in the main region of display 12. When the ‘Events’ page is displayed, key 24 is shown in a pressed state. ‘Events’ key 24 is preferably always enabled. Events may be categorized into events such as ‘Bypass/ECMO’, ‘Fluid’, and ‘Manual Set Point’ events. Events and timers are displayed in a status bar as described in detail below. ‘Bypass/ECMO’ events are saved and may be both automatically and manually recorded, and recordings may be started or stopped by pressing an assigned key, such as a key displaying the software logo. Other keys are customizable from within the setup mode, and new items require naming, selecting whether the event is a timer event, and if selected as a timer event, then selecting ‘time on’ or ‘time on and off’ for the event. ‘Fluid’ events include entering a name and non-zero numeric value. Entering a value adds an event to the event list and fluid balances are recomputed from entered values. A ‘Cardioplegia’ event key is also preferably included, and new ‘Manual Set Point’ events may be added.

The cardioplegia event key is shown depressed in the ‘off’ state. Pressing the cardioplegia key prompts the system to display a further set of keys, such as an ‘Alarm’ key. Pressing the ‘Alarm’ key opens a numeric keypad with memory slots for inputting alarm times in units of minutes. The alarm limit is the interval after cardioplegia is stopped until the field in the status bar provides a visual indication, such as blinking of the timer. The cardioplegia list includes ‘Add’, ‘Remove’, ‘Page’, ‘OK’, and ‘Cancel’ keys. The ‘OK’ key starts cardioplegia and is enabled when the case is running. ‘Remove’ is enabled when the case is not running and in setup mode. Thus, the two keys may share the same position.

‘Event Chart’ key 26 opens an ‘Event Chart’ page in the main region of display 12. When the ‘Event Chart’ page is displayed, key 20 is shown in a pressed state. ‘Event Chart’ key 26 is enabled once an event has been entered. The ‘Event Chart’ page is an events summary page where the events recorded during a case are summarized. Events may be edited and removed. Events are added through other pages as described above and below. The event summary in an exemplary embodiment is displayed as a table with data such as event, time, value, fluid and comments. A ‘Remove’ function is enabled once an event is entered into the table. Removal of an item requires confirmation of a removal event. Events may be searched or arranged by type.

Exemplary event types include, but are not limited to, ‘Red Flag’, ‘Bypass/ECMO’, ‘Fluid’, ‘Manual Set Point’, ‘Cardioplegia’, ‘Blood Sample’, ‘Personnel Change’, ‘Disposable Change’, and ‘Equipment Change’ events. ‘Add Event’ key 28 is pressed to cause a ‘Red Flag’ event to be added at the current time, kept by the monitor as described below, to the event list. ‘Add Event’ key 28 is enabled when the system is in the ‘Started’ or ‘Stopped’ states. Under add event, a user is able to edit name, time, value, fluid volume and comment, for example. Until a name has been entered, the name cell is populated with an icon, such as a flag. All fields are editable and enabled for red flag events. Event values in the fluid field are included in fluid balance. ‘Bypass/ECMO’ events are added as described above. Value fields are either ‘On’ or ‘Off’ for timer events, or blank and disabled for non-timer events. The remainder of the events are added as described above.

‘Cardioplegia’ key 30 opens a ‘Cardioplegia Summary’ page in the main region of display 12. When the ‘Cardioplegia Summary’ page is displayed, key 30 is shown in a pressed state. The ‘Cardioplegia Summary’ page is where cardioplegia events recorded during a case are summarized. Events may be edited and removed through the page. Events are added as provided above. Editable fields include ‘Time On’, ‘Time Off’, ‘Duration’, and ‘Volume, ml’, for example. When cardioplegia is ‘On’, then the time off, duration, and volume fields are disabled and blank. A ‘Remove’ functionality with confirmation is also provided for removing an event.

‘Blood Sample’ key 32 opens a ‘Blood Sample’ page in the main region of display 12. When the ‘Blood Sample’ page is displayed, key 24 is shown in a pressed state. ‘Blood Sample’ key 32 is enabled when the system is in the ‘Started’ or ‘Stopped’ states, or whenever setup mode is enabled. The ‘Blood Sample’ page is provided for the user to enter blood samples taken such as blood gases and Activated Clotting Time (ACT) tests. The page may include fields for types and results. Selecting a type adds a new event. Results may include types and time and event occurred.

‘Setup’ key 34 opens a ‘Set-up’ page including a ‘Password’ key for allowing the application to enter set-up mode, as well as a plurality of sub-keys for accessing sub-pages. Sub-keys and corresponding pages include ‘Policies’, ‘Input Configuration’, ‘Template’, ‘Network Status’, ‘Chart Configuration’, and ‘Statistics’. When the ‘Setup’ page is displayed, key 34 is shown in a pressed state. ‘Setup’ key 34 is enabled when the system is in the ‘Started’ or ‘Stopped’ states.

The ‘Policies’ sub-page is provided for selecting formulas, units, record number format, date information, patient table names, computations, event group labels, modes locations, among other functions. The ‘Input Configuration’ sub-page is provided for setting up the cardioplegia, blood gas and continuous data tables. The user is further able to define how data maps from devices to fields in the chart, how manual input data is formatted, and control limit values.

The ‘Template’ sub-page provides a complete set of settings for the cardioplegia, blood gas and continuous data. Selecting a template resets the configuration information. If the configuration is the same as a template, the key is shown pressed. Changing a value in the grid causes the key to display as depressed. Pressing the ‘Add’ key on the template control prompts the user to input a character-based label. The settings shown in the sub-page will be the template settings. Mode, i.e. ‘Cardioplegia, ‘Blood Gas’ or ‘Continuous’ may be changed and is shown in the title of the page. Configuration grids are setup by adding a label, locations defining where data comes from to populate a chart, and low/high control limits (e.g. one set for cardioplegia and continuous, and two sets for blood gas corresponding to arterial and venous.

The ‘Network Status’ sub-page displays to the user all connected devices and does not require the user to be in setup mode to view the sub-page. The ‘Chart Configuration’ sub-page contains options for the format of the output chart, including margins, page size, table dimensions and data recording frequency. The ‘Chart Configuration’ sub-page further provides fields for entering the hospital name, address and chart title. The ‘Statistics’ sub-page includes selectable categories such as ‘Time’, ‘Fluid’ and ‘Parameter’, and corresponding statistics, and displays currently added statistics for the selected category.

‘Records’ key 36 opens a ‘Records’ page in the main region of display 12. When the ‘Records’ page is displayed, key 36 is shown in a pressed state. ‘Records’ key 36 is preferably always enabled. The ‘Records’ page is provided for displaying the current chart for review and may be displayed during a case and updated as new data arrives. A list of bookmarks are provided for directing the user to the corresponding section of the chart. Bookmarks include items such as, but not limited to, ‘Print’. ‘Cardioplegia’, ‘Checklist’, ‘Continuous Data’, ‘Disposables/Equipment’, ‘Event Chart’, ‘Patient Data’, ‘Personnel’, ‘Pre-Op Labs’, ‘Priming Fluids’, ‘Priming Statistics’, ‘Signatures’, and ‘Statistics’.

‘Software’ key 38 causes the monitoring software to be displayed overlaying the monitoring system application. Software key 38 is preferably always enabled.

### Control Limit Notification

“Control Limits” are defined herein as the ranges for a parameter that the surgeon or other has identified as acceptable ranges for the case. If a parameter falls outside of the predeteimined limits, an event is created as described above and all parameters are reported at that time. Once a control limit notification is provided, the user should revisit the event and enter comments to detail the cause of the condition or to make any relevant notes. This is achieved using the ‘Event Chart’ function as described above. Control limits are chosen by selecting a template using the ‘Personnel’ page in the ‘Operation Manager’ as described above. The control limits are entered in the ‘Setup/Input’ configuration as described above. When a value falls outside of the parameter control limit, a control limit notification such as a pop-up window is displayed. The popup includes a list of the control limit events, the time, which parameter the event applies to, and whether the value was above or below the limits. The popup remains until the user closes the window. The control limit popup feature includes a ‘Snooze’ for disabling popups for a predetermined period of time.

### Status Bar

Referring again to FIG. 1, status bar 50 includes event timers 52, 54 and cardioplegia timer 56, as well as fluid balance 58 and system clock 60 displays. System clock 60 keeps the current system time in an hr:min:sec format. The clock and timer strings are preferably updated at 1 s intervals. Timers 52, 54 and 56 display information and track time corresponding to an event. At least timer 56 may additionally blink in response to an alarm as a visual alert to the user. Event strings are preferably formatted based upon the current status of the timer with the option for the timer to display only the ‘Time on’, or both the ‘Time on and time off’. For an event that has not yet started, the string remains empty. For a ‘Time on’ only event following another ‘Time on’ only event, the timer string may be displayed in the form of:

<Event Name> On at <TIME> (<DURATION>)

<Event Name> - name of the event
<TIME> - clock time when event was turned on in hr:min format
<DURATION> - time the event has been on in min:sec format

If the event is a time on and off event, the timer string may be displayed as:

<Event Name> <State> at <TIME> (<DURATION>)

<Event Name> - name of the event
<State> - last state of the event (‘On’ or ‘Off’)
<TIME> - clock time of last event in hr:min format
<DURATION> - time of the event since last event in min:sec format

Fluid Balance is preferably displayed at 58 and may be formatted as follows:

<sign><X><units>” where <sign> will be ‘+’ or ‘−’ (with positive indicating fluid going into the patient), <X> being the fluid balance in ml.

Fluid balance may be computed as follows:

Fluid Bal=ΣPriming Fluids+ΣFluids added−ΣFluids removed+ΣCardioplegia

### Configuration Saving

In one embodiment, to preserve data, items may be added at any time while items may only be removed when the system is in ‘Setup’ mode. In an alternative embodiment, certain items may be removed when the system is in other modes. Changes to item lists are preserved for future sessions when a case is closed in ‘Setup’ mode or if the machine is shutdown in ‘Setup’ mode. User configuration options and user input history are preferably saved as ASCII text files, and file names preferably have the extension of “.dat” with the first 4 bytes being a CRC checksum of the rest of the file, as known to those skilled in the art.

While the preceding cardiac monitoring systems and methods have been described with reference to specific embodiments and examples, it is envisioned that various details of the invention may be modified without departing from the spirit and scope of the invention. Furthermore, the foregoing description of the preferred embodiments of the invention and best mode for practicing the invention are provided for the purpose of illustration only and not for the purpose of limitation.

What is claimed is:

1. A method for configuring a case template in a cardiac monitoring system, comprising the steps of:
selecting critical parameters to be monitored during a procedure;
entering patient-specific details for computing patient values for determining control limits for the critical parameters; and
setting control limits for each of the selected critical parameters.

2. The method according to claim 1, further comprising the steps of:
entering names of personnel attending the procedure;
entering pre-operative lab values for the patient;
entering priming-related data for a priming circuit for the procedure and calculating optimal fluid compositions based on the patient-specific details and pre-operative lab values;
entering disposable items to be used during the procedure;
entering equipment to be used during the procedure; and
configuring event timers for the procedure corresponding to parametric data falling outside of the set control limits.

3. The method according to claim 2, wherein the patient values include at least one of body mass index, body surface area, body surface area cardiac index, weight cardiac index, body surface area flow rate, and weight flow rate.

4. The method according to claim 2, wherein the step of entering priming-related data includes selecting bypass factors for loading preset priming volumes and computing drug volumes from dosages.

5. The method according to claim 1, further comprising the steps of:
monitoring parametric data in real-time against the set control limits;
providing notification of control limit breach events;
capturing and recording event parametric data related to the control limit breach events;
charting the event parametric data corresponding with the timing of the control limit breach events;
producing a perfusion chart record including the event parametric data; and
saving the template according to an appropriate clinical or control profile.

6. The method according to claim 5, wherein the monitored parametric data includes both static and continuous data, wherein static data is data provided to the monitoring system on an intermittent basis and continuous data is data collected at a user specified frequency.

7. The method according to claim 5, further comprising the step of providing a graphical user interface including a display for displaying a plurality of soft keys for accessing a plurality of data pages each for at least one of entering, receiving, storing, arranging, manipulating, charting, generating, retrieving, viewing and reporting data.

8. The method according to claim 5, further comprising the steps of communicating with a hospital management system and downloading the perfusion chart record to a database for study to improve quality of care.

9. The method according to claim 5, further comprising the step of categorizing the control limit breach event into one of a bypass, fluid, manual set point, cardioplegia, blood sample, personnel change, disposable change, equipment change, and flagged event.

10. The method according to claim 5, further comprising the step of providing a checklist for confirming that a set of predefined tasks have been completed prior to initiating monitoring the parametric data, wherein the predefined tasks include clock synchronization, entry of the patient-specific details and equipment checks.

11. The method according to claim 1, wherein the critical parameters are selected according to surgeon preferences, patient details and procedure type.

12. A cardiac monitoring system, comprising:
a graphical user interface for displaying a plurality of soft keys for accessing a plurality of data pages each for at least one of entering, receiving, storing, arranging, manipulating, charting, generating, retrieving, viewing and reporting data; and
a storage device including a computer-readable medium containing instructions for configuring a cardiac monitoring procedure template that includes configuration functions for selecting critical parameters to be monitored during the procedure, entering patient-specific details for computing patient values for determining control limits for the critical parameters, setting control limits for each of the selected critical parameters, monitoring parametric data in real-time against the set control limits, providing notification of control limit breach events, capturing and recording event parametric data related to the control limit breach events, charting the event parametric data corresponding with the timing of the control limit breach events, and producing a perfusion chart record including the event parametric data.

13. The system according to claim 12, wherein the configuration functions further include at least one of saving the template according to an appropriate clinical or control profile, entering names of personnel attending the procedure, entering pre-operative lab values for the patient, entering priming-related data for a priming circuit for the procedure and calculating optimal fluid compositions based on the patient-specific details and pre-operative lab values, entering disposable items to be used during the procedure, entering equipment to be used during the procedure, and configuring event timers for the procedure corresponding to parametric data falling outside of the set control limits.

14. The system according to claim 13, wherein entering priming-related data includes selecting bypass factors for loading preset priming volumes and computing drug volumes from dosages.

15. The system according to claim 12, wherein the patient values include at least one of body mass index, body surface area, body surface area cardiac index, weight cardiac index, body surface area flow rate, and weight flow rate.

16. The system according to claim 12, wherein the monitored parametric data includes both static and continuous data, wherein static data is data provided to the monitoring system on an intermittent basis and continuous data is data collected at a user specified frequency.

17. The system according to claim 12, further comprising a hospital management server in communication with the cardiac monitoring system for downloading the perfusion chart record thereto to improve quality of care.

18. The system according to claim 12, further comprising the configuration function of categorizing the control limit breach event into one of a bypass, fluid, manual set point, cardioplegia, blood sample, personnel change, disposable change, equipment change, and flagged event.

19. The system according to claim 12, further comprising the configuration function of providing a checklist for confirming that a set of predefined tasks have been completed prior to initiating monitoring the parametric data, wherein the predefined tasks include clock synchronization, entry of the patient-specific details and equipment checks.

20. The system according to claim 12, wherein the critical parameters are selected according to surgeon preferences, patient details and procedure type.
